(19) United States
(12) Patent Application Publication
(10) Pub. No.: US 2024/0207525 A1
(43) Pub. Date: Jun. 27, 2024

(54) CONTAINER FOR EJECTING A SUBSTANCE AND RELATED PRODUCTION METHOD

(71) Applicant: 3CK S.R.L., ARZIGNANO (IT)

(72) Inventors: Angelo CONSOLARO, ARZIGNANO (IT); Roberto CONSOLARO, ARZIGNANO (IT); Rajeev KABBUR, ARZIGNANO (IT); Ajay KABBUR, ARZIGNANO (IT); Edoardo CONSOLARO, ARZIGNANO (IT); Francesco Federico CONSOLARO, ARZIGNANO (IT); Massimo CONSOLARO, ARZIGNANO (IT)

(21) Appl. No.: 18/573,972

(22) PCT Filed: Jun. 24, 2022
(86) PCT No.: PCT/IT2022/050180

§ 371 Date: Dec. 22, 2023

(30) Foreign Application Priority Data
Jun. 24, 2021 (IT) 102021000016637

(51) Int. Cl. A61M 5/28; A61M 5/50

(52) U.S. Cl. CPC A61M 5/282; A61M 5/50; A61M 2207/00

(57) ABSTRACT

A container for ejecting a substance, such as medicaments and the like, the container including a hollow shell for the containment and ejection of the substance, and a membrane connected to the shell and having a rim. Advantageously, the container further includes a shaping element for connecting to said membrane so as to switch from a first operating position to a second operating position. A method for producing said container is also disclosed.

### CROSS-REFERENCE TO RELATED APPLICATIONS

The present application claims the priority of international application No. PCT/IT2022/050180, filed Jun. 24, 2022, and which claims the priority of Italian application No. IT102021000016637, filed Jun. 24, 2021, the disclosures of which are incorporated by reference herein in their entireties.

### BACKGROUND

### Technical Field

In more detail, the application relates to a container for ejecting a substance, the structure of which is such as to maintain the trade-off of integrity between the parts of the container and the “break-loose force” for the functioning of the container, further, the method of use and production thereof is such as to enable the creation of products with different uses and purposes operating under the same principle according to the present application.

### Description of Related Art

In the medical sector, devices for dispensing substances such as, by way of example only, conventional syringes and pre-filled syringes are widely used.

Generally, a conventional syringe consists of a cylinder containing the substance to be dispensed, a plunger to create pressure to push the liquid and a plunger to push the substance out of the cylinder, and an opening or cannula to allow dispensing. The performance of syringes with a plunger depends on a good compromise between the trigger force and the hydraulic seal between the plunger and the cylinder. In particular, by way of example, this type of seal is designed not to allow any substance or gas to pass between the plunger and the cylinder; furthermore, the seal itself is affected by the interference between the diameters of the cylinder and those of the plunger.

In detail, an initial problem with the above-mentioned type of syringe is the fact that a greater interference between the piston diameter and the cylinder diameter results, on the one hand, in a better integrity of the syringe, but on the other, in an increase in the trigger force and movement of the syringe. For this reason, in all types of syringes, whether made using the ‘Blow Fill Seal’ (BFS) procedure, or otherwise, lubricants such as silicone oils are used to reduce the friction force and improve the seal of the piston with the cylinder in the syringe.

In particular, the performance of the syringe depends, primarily, on the coefficient of friction between the two elements: piston-cylinder, the amount of lubricant (e.g. silicone oil) present on the piston and/or the lubricant applied to the internal surface of the cylinder.

A further problem lies in the fact that, for example, in a pre-filled syringe, the lubricant that remains in contact with the substance contained in the cylinder, especially when said substance is a drug, could potentially cause aggregates that are highly undesirable because they could contaminate and thus reduce the efficacy of the drug itself.

Nowadays, in an attempt to overcome the aforementioned problems, what is done is to seek a compromise between the amount of lubricant used and the interference surface area between the cylinder and the piston, or to produce pistons and/or cylinders coated with an additional material, with the aim of reducing friction and contamination. However, in the latter case, in order to place the coating on the piston and/or cylinder, it is necessary to introduce additional steps in the production process, which increases costs; furthermore, the new coating material, coming into contact with the substance contained in the cylinder, may also cause contamination and/or undesirable effects.

Not the least of the known state of the art problems is that, for example, in a pre-filled syringe with piston and cylinder, it is important that the ovality of the cylinder diameter is contained within certain limits. If the ovality of the cylinder is greater than these limits, then the piston-cylinder interference must be increased; similarly, the manufacturing process of the cylinder involves a certain tapering of the cylinder which causes problems with piston-cylinder interference. In detail, this step to reduce the ovality and taper of the cylinder leads, disadvantageously, to a more expensive manufacturing process.

Finally, even a traditional cylinder-piston syringe, if subjected to temperature differences and/or excessive pressure, could also be more likely to lose integrity between the piston and cylinder.

At present, there is no container on the market for the ejection of a substance that is economical but, at the same time, allows a compromise between the integrity of the system and the trigger force.

Furthermore, there are currently no known mechanisms in the industry that also guarantee a delivery of substance in the correct quantity.

### SUMMARY

In the context of the above-mentioned requirements, therefore, the main scope of the present application is to propose a container for the ejection of a substance, for example of one or more certain medicaments, capable of obviating the technical drawbacks mentioned above and, in particular, to realise a container which guarantees safety in use, whatever its form.

A further object of the present application is to realise a container for ejecting at least one substance, which guarantees a compromise between the trigger force and the integrity of the system, irrespective of possible variations in temperature and/or pressure.

An additional object of the present application is to make a container for ejecting at least one substance which is such as to ensure complete delivery of the substance and, if necessary, to ensure non-reusability by the user himself or by third parties.

A further object of the present application is to realise a container for the ejection of a substance which allows safe use in several respects: hygiene, precision in drug delivery and intrinsic safety for the patient and the operator.

Further, it is an object of the present application to provide a container, which can be used for making conventional, pre-filled syringes, vials, or other medical or non-medical containers.

It is further an object of the present application to make a container which is functional, of rapid implementation and sufficiently precise in releasing the medication.

The object of the present application is thus to create a product which is cost-effective, easy to manufacture, useable and safe.

These and other purposes are achieved by a container for ejecting a substance according to the application, as will become clear later in this description.

It is therefore an object of the present application a container for ejecting a substance, such as medicaments and the like.

In particular, the container object of the present application comprises a hollow shell for the containment and ejection of the substance and including a containment portion with a dispensing organ of oblong shape, to facilitate the ejection of the above-mentioned substance;
moreover, the container also comprises a membrane, connected to the same shell.

Advantageously, the container comprises, in a position contiguous to and transverse with respect to the containment portion, a shaping, for connecting the membrane to the shell.

In an advantageous way, said membrane connected to the shaping of the shell is so that it can move from a first operating position, in which the membrane has a central portion in a position opposite to the containment portion of the shell, so as to create, with the containment portion, a chamber for the containment of the substance, to a second operating position, wherein the central portion of the membrane collapses within the portion of containment of the shell, so as to eject the substance present in the chamber through the dispensing organ.

Additionally, the membrane is calibrated with the geometry of the containment portion of the shell, so as to adhere to and be compressible in the direction of same shell, for ejecting the substance contained in the chamber.

Advantageously, the membrane is of the bi-stable type so as to independently maintain its own operating position between the operating positions described above.

In particular, such a membrane does not require any tools such as, for example, a plunger to maintain its position while using the container.

In addition, depending on the thickness of the membrane, both the intensities of a compressive force applied on the membrane and said bistability vary.

Moreover, the membrane has the form of a prolate or oblate spheroid or other form suitable for containing, advantageously, the collapse of the same into the shell.

Always in an advantageous way, the shell comprises a set of sharp-edged notches for perforating the membrane, in particular when in the second operating position, for ejecting the substance comprised in the container.

In addition, the shell also comprises grooves, facing in the direction of the chamber, to facilitate the ejection of the substance from the same chamber in which is contained.

Advantageously, the dispensing organ of the containment portion of the shell is connected to dispensing accessories such as, for example, needles.

Moreover, the shell comprises a support section for the fingers of a user.

In an advantageous way the container also comprises a movable activator device, having a shape corresponding to the shape of said shell, having a head adapted to push and compress said membrane inside the shell, in order to pass from the first operating position to the second position, and a body adapted to transmit the compression on the membrane during the use of the container.

Advantageously, the head of the activating device comprises a plurality of concentric slots for relieving it and for regulating the force necessary for deformation of the membrane.

Moreover, the head comprises cavities for facilitating the perforation of the membrane, by means of said sharp-edged notches present on the inner surface of the shell, preventing, in an advantageous way, the reuse of the container.

The head of the activator device also comprises sharp-edged notches capable of perforating the membrane after the relative compression, preventing reuse of the container.

In an advantageous way, the activator device comprises a toothed profile along the body wherein the pitch between the teeth of said toothed profile corresponds to a unit volume of the substance to be ejected.

In addition, the membrane and the activator device comprise, respectively, a coupling and a hole such that the coupling locks within the hole, to enable the membrane to be brought to the first operating position and to aspirate the substance into the chamber of the shell.

The membrane comprises also a protrusion, facing in the opposite direction of said coupling, the protrusion being apt to expel any substance remaining in the dispensing portion of the container.

Advantageously, the container comprises a ring for locking the membrane to said shell, wherein the ring is coupled to the shell and is apt to accommodate within it the activator device guiding its movement.

The shell comprises also a first portion tubular and hollow contiguous to the containment portion, wherein said ring is coupled, so as to fit within the first portion of the shell.

The membrane, comprises also an edge including a transverse portion terminating in a first apex, contiguous with and transverse to said transverse portion.

In further embodiments, the membrane comprises a second apex, contiguous and transverse to the transverse portion, wherein the second apex is adjacent and facing in the same direction as the first apex.

Moreover, the membrane comprises a third apex, transverse to the transverse portion, and positioned between the first and second apexes and facing the opposite direction from the same, to improve the tightness of the membrane.

The activator device comprises, advantageously, one or more rings for preventing the reuse of the container after use, preventing the return of the container to the first operating position.

In an advantageous way, the container also comprises a projecting element, suitable for switching from a rest position corresponding to the first operating position to an activation position corresponding to the second operating position in which, the projecting element is engaged with at least one of the one or more rings of the activating device, so as to secure it to the shell, to prevent its reuse.

The ring comprises, in preferred but not limiting embodiments, a projecting segment of a shape substantially complementary to said transverse portion of the membrane, and facing inwardly of the chamber, suitable for locking said transverse portion of the membrane at the shaping of the shell by holding it in place and acting as a seal.

Moreover, the ring comprises in a position contiguous with and opposite to the projecting segment a complementary flat segment and such as to contact the apex of the membrane.

Advantageously, the projecting segment and the flat segment of the ring are apt to respectively press the apex and the transverse portion of the membrane within the shaping of the shell.

In addition, the ring comprises, in a position opposite to the projecting segment a support preferably for the fingers of a user.

In an advantageous way, object of the present application is also a production method of a container as described above, comprising the following steps:
A. Making the parts of the container, by one or more of the following processes: blow molding, injection molding, compression molding, BFS, thermoforming or 3D printing wherein the parts comprise the shell, the membrane, the ring and the activator device;
B. Assembly of said parts in a suitable, sterile environment;
Secondary packaging of the container in a suitable environment;
C. Terminal sterilization of the container using one or more of the known methods of sterilization.

Advantageously, when in the production method the step of making the parts of the container, takes place by means of the injection and/or compression molding technique, during the production process, there is a step of controlling the cooling process of the parts so that the shaping and the transverse portion have a higher temperature than the rest of the membrane and the rest of the shell, so that the membrane sticks to the shell without the use of additional instrumentation.

Always in advantageous way, when in the production method the step of making the parts of the container, is carried out by means of the “Blow Fill Seal” (BFS) technique, during the manufacturing process, a portion of the shell is molded with a greater thickness than the remaining portion of the shell so as to make the shell rigid and a portion of the membrane with a lower thickness than the remaining portion of the membrane so as to make said membrane more flexible.

Moreover, when the step of making the parts of the container, takes place by means of the “Blow Fill Seal” (BFS) technique, during the production process, and before closing a mold, a shell-shaped insert is added to the container, wherein it comprises a containment portion and a dispensing organ.

In addition, when the step of realizing the parts of the container, takes place by means of the “Blow Fill Seal” (BFS) technique, during the production process, an insert in the form of a membrane suitable for being incorporated and/or welded on the shaping of the shell is added to said container, when still malleable.

Moreover, during the phase of realization of the parts of the container, when the moulding technique is used, there are the following further phases

As the arrangement of a plurality of shells within a molding die using a suitable nest for the arrangement of the shells in which, a heat-sealable film is positioned against the top of the shells being made,

Heating the edge of the shell and the film by a methodology and/or a combination of several methodologies such as, for example, induction, hot air blowing, use of infrared lamps, by convection;

Pressure of a mold against the molding die of the still malleable shells such that the film is welded to the molding of the shell;

Application of pressure difference at the shell and/or pressure at the delivery organ, on the molding die, such that, advantageously, the film deforms to form the relatively flexible membrane.

In addition, during the step of assembling the parts of the container the ring is positioned in such a way as to exert pressure between the transverse portion of the membrane and the shaping of the shell.

Advantageously, during the step of assembling the parts of the container, a layer of glue is arranged between the shaping of the shell and the portion of the membrane, to maintain the seal.

Moreover, during the step of assembling the parts of the container, in order to achieve a tight seal between the shaping and the transverse portion, the same are heated and pressed together by the application of an external pressure.

Advantageously, always during the step of assembling the parts of the container, ultrasound or infrared or other techniques known to promote mechanical engagement and interference between the parts are used to weld the portion of the membrane with the shaping of the shell.

In addition, prior to use, the container is filled with the substance and the toothed profile of the activator device is adapted to compress the membrane, eliminating the air present in the chamber of the container, so that the activator device locks in the first operating position.

### BRIEF DESCRIPTION OF THE DRAWINGS

Further features and advantages of the container for ejecting a substance according to the present application will be more apparent from the following description, referring to a preferred and illustrative, but not limiting, embodiment and the accompanying drawings, wherein:

FIG. 1A is a frontal sectional view showing the container for ejecting a substance in a first operating position wherein the container is ready for use, then before use thereof;

FIG. 1B is a frontal sectional view showing the container of FIG. 1A, in a second operating position, when in use;

FIG. 1C is a frontal sectional view showing the container of FIG. 1A, in a third end-use position;

FIG. 1D is a detail view of the container of FIG. 1A in a first position of beginning use;

FIG. 1E is a view of a detail of the container of FIG. 1C in an end-use position;

FIGS. 2A to 2D show frontal and sectional views of further embodiments of the container membrane;

FIG. 2E shows a further embodiment of the membrane and container;

FIG. 2F shows a detail relating to an improved system for sealing the membrane in the container;

FIG. 3A shows a detail of the container with evidence of any disorderly states;

FIG. 3B shows a detail relative to an embodiment of the shell belonging to the container;

FIG. 4A shows a perspective view of a further embodiment of the container;

FIGS. 4B to 4E show details of an embodiment relating to the container of FIG. 4A;

FIGS. 5A and 5B show a frontal sectional view of a further embodiment of the container in two different positions of use;

FIGS. 6A and 6B show a frontal sectional view of a further embodiment of the container in the positions of initial use and end of use respectively;

FIG. 6C shows a detail of the section depicted in FIG. 6B;

FIGS. 7A-7C show frontal and sectional views of a further embodiment of the container;

FIGS. 8A and 8B show frontal and cross-sectional views of a further embodiment of the container in the initial and mid-use positions, respectively;

FIG. 8C show a detail of the sectional view depicted in view 8A;

FIGS. 9A and 9B show one of the methods for producing the container;

FIGS. 10A to 10C. show graphs depicting different trends in the deformation force of the container;

FIGS. 11A and 11B show a front sectional view of a further embodiment of the container;

FIG. 12A shows a perspective view of a further embodiment of the container formed by a blow-fill-seal (BFS) process;

FIG. 12B shows a front sectional view of the container of FIG. 12A;

FIG. 12C shows a graph of the thickness control of the container parts, used by the BFS process of FIG. 12A;

FIGS. 13A-13C show frontal sectional views of three further embodiments of the container formed using the BFS process with inserts.

### DETAILED DESCRIPTION

With reference to Figures TA to 1C, a first embodiment of the container for ejecting a substance, generically referred to by the numerical reference 100, is observed.

The container 100, in preferred but not limiting embodiments, is a syringe suitable for the containment and ejection of at least one substance, in particular medicaments.

Advantageously, in contrast to the related art described above, the container 100 object of the present application does not have any piston for dispensing the substance and thus becomes more reliable.

Advantageously, the container 100 has a very simple design comprising a hollow shell 1 for the containment and ejection of a substance and a deformable membrane 2, wherein said shell 1 is substantially more rigid than said membrane 2 mentioned above.

Further, the container 100 comprises, in preferred but not limiting embodiments, a plunger 3 capable of deforming the membrane 2 for ejection of the substance and a ring 4 for locking said membrane 2 to the shell 1, wherein the ring 4 is also capable of guiding the movement of the plunger 3 within the container 100.

The shell 1, as visible in the figures, comprises a first portion 1.2 of preferably tubular and hollow shape and a second containment portion 1.1 of concave, which is contiguous with and has a smaller diameter than the first portion 1.2.

As can be seen in the figures, the second portion 1.1 of the shell 1 has a substantially “cup” shape, e.g. hemispherical, ending, at the central/base point of the “cup”, with a dispensing organ 1.1.1. Still advantageously, in further embodiments of the container 100, said dispensing organ 1.1.1. can be arranged in an off-center position with respect to the central point/base of the “cup”, in particular at the side of the shell 1.

Advantageously, the dispensing organ 1.1.1 is contiguous to the second containment portion 1.1 and is hollow and of oblong shape, to allow dispensing of the substance contained in the container 100 itself.

Advantageously, the containment portion 1.1 and, in particular, the associated dispensing organ 1.1.1, in preferred, but not limiting, embodiments, is connected to accessories, such as a needle, for dispensing the substances contained within the container 100. Furthermore, the dispensing organ 1.1.1, prior to use of the container 100, is capped by a cap (not visible in the illustrated embodiments), which cap is removed prior to use allowing, if necessary, any accessories for dispensing the substance such as a needle or connector to be attached.

Further, between the first portion 1.2 and the second portion 1.1 the container 100 advantageously comprises a shaping 1.6 connecting the two portions, so that the second portion 1.1 is projecting internally, by a section 1.4 of the shell 1, with respect to the first portion 1.2. Advantageously, said section 1.4 goes from a first edge facing inwards of the container 1, to a second edge facing outwards.

In particular, the shaping 1.6 results in a surface that is contiguous and transverse with respect to the first portion 1.2, wherein the second edge of the shaping 1.6 has substantially the same inner diameter as the first portion 1.2.

Advantageously, a membrane 2 is placed at the surface of the aforementioned shaping 1.6.

Advantageously, since the shell 1 can be made by means of different manufacturing techniques such as, by way of example only, blow moulding, moulding, injection moulding, compression moulding, BFS, thermoforming or 3D printing, it is made of plastic or substantially elastic and/or plastic materials.

Again with reference to the embodiment under consideration, the membrane 2 is substantially concave and made so as to be deformable, turnable and/or bi-stable. By bi-stable, it is meant that the membrane 2 maintains position independently and therefore does not require the plunger 3 to be held in position or to be squeezed to match the shell 1, as will be explained below.

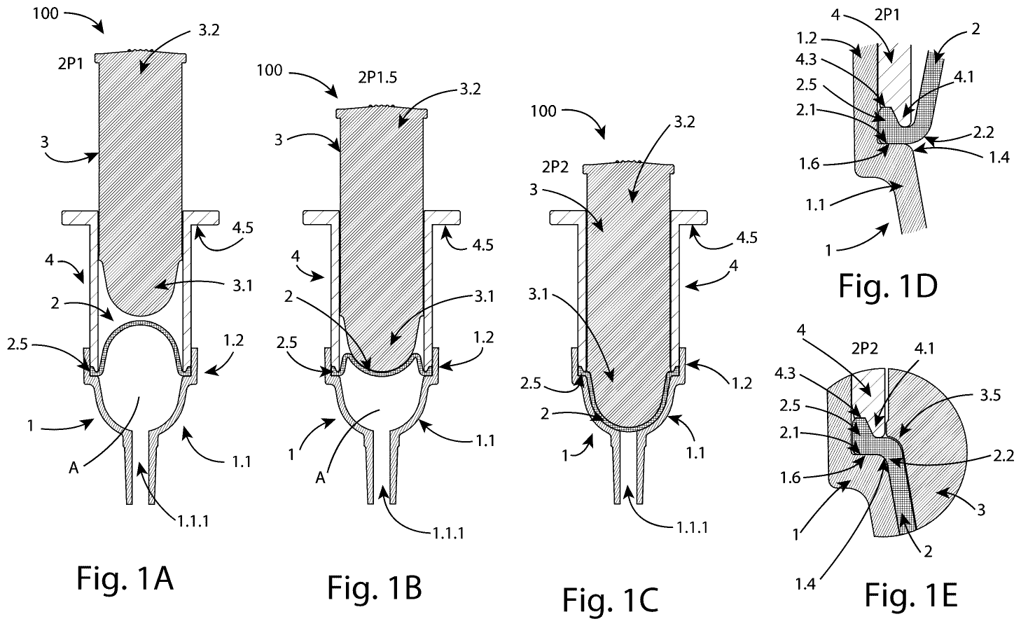

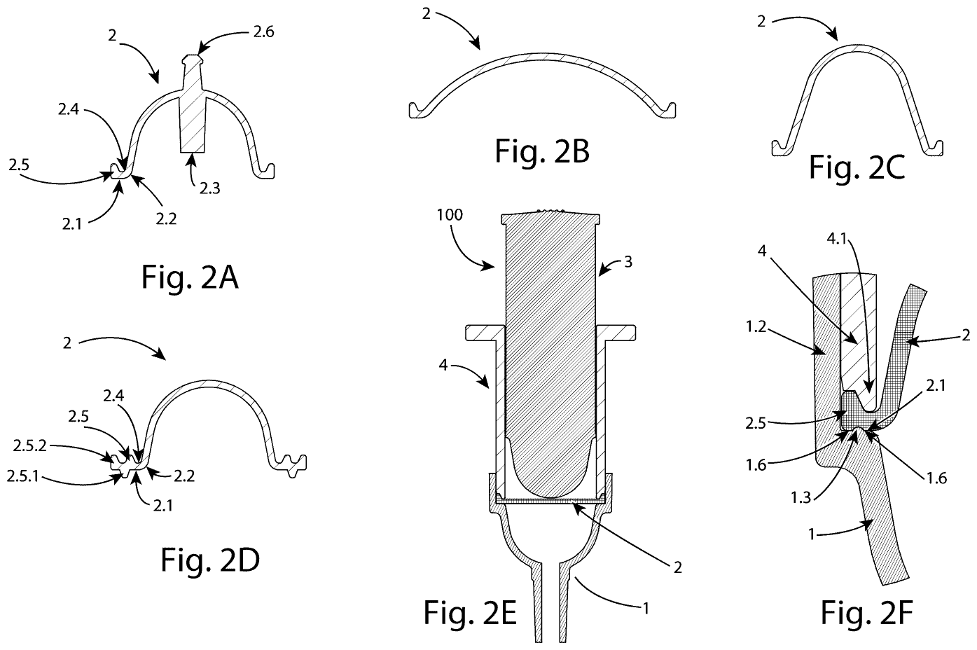

In detail, in the embodiment under consideration, the membrane 2 has a substantially prolate spheroid shape and is connected to the shell 1, as visible in FIGS. 1A, 1B and 1C, in correspondence with the relative shaping 1.6. Advantageously, said membrane 2 may have, as required, other shapes or configurations such as, by way of example only, those depicted in FIGS. 2B and 2D, wherein the same has a shape substantially of a prolate spheroid or an oblate spheroid.

Advantageously, similarly to the shell 1, the membrane 2 is made of elastic and/or plastic materials and is shaped in such a way that the shape of the shell 1 coincides with that of the membrane 2 to allow, as will be seen later, a complete dispensing of what is contained in the container 100.

Advantageously, the membrane 2 may be circular, rectangular, square or any shape, whether regular or irregular. In detail, the shape of the membrane 2 is such that it advantageously influences the operation of the container 100 in its entirety, e.g. in the case where the membrane 2 is designed in the shape of a prolate spheroid, as visible in FIG. 2C, the force required for its compression in the direction of the shell 1 and, therefore, the force to be applied on it by the plunger 3 will be greater than in the case where the membrane 2 is designed in the shape of an oblate spheroid (FIG. 2B).

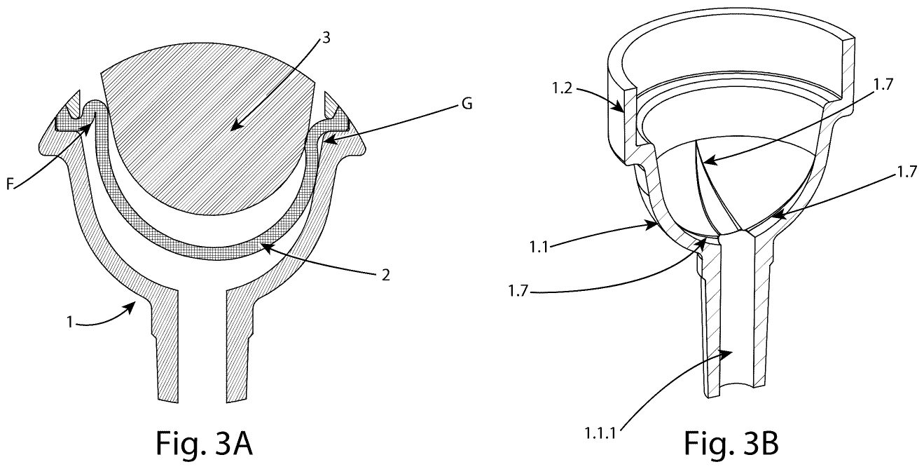

In particular, factors such as the thickness, elasticity, hardness and tensile strength of the membrane 2 are physical properties that determine the possible formation of folds F on the surface of the membrane 2 itself and/or possible gaps G (FIG. 3A) formed during the deformation process of the membrane 2 towards the shell 1 by the plunger 3.

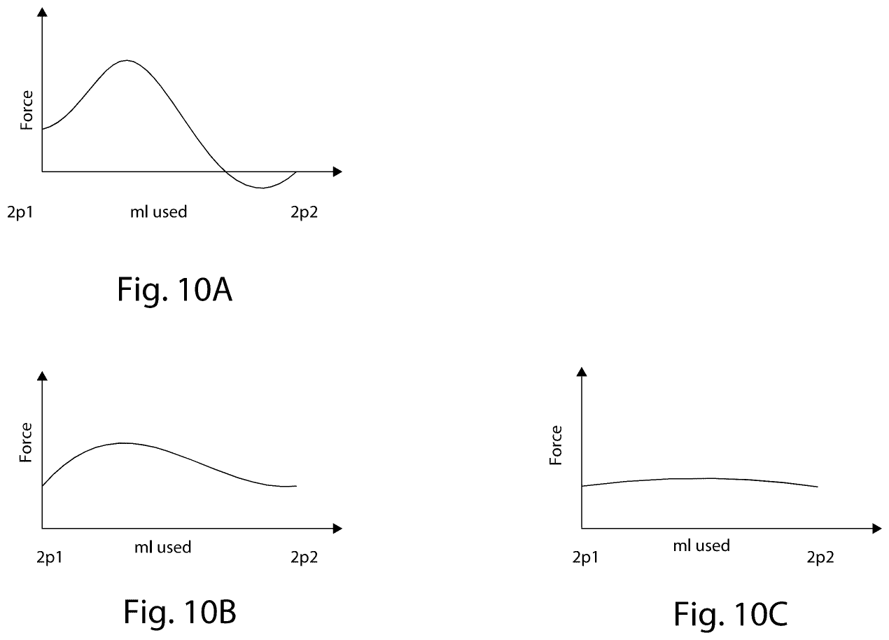

If Membrane 2 is made so that it is thicker at its central area, so that the thickness gradually decreases as it moves away from the centre, the force required to compress it will be almost constant (FIG. 10C); conversely, if Membrane 2 is uniformly thick and rigid, the force profile to move Plunger 3 will not be as constant (FIG. 10A-10B).

Further, as visible in FIGS. 1D and 1E, said membrane 2 comprises a transverse portion 2.1 in correspondence with the relative edge of the concavity, which transverse portion 2.1 terminates with an apex 2.5, contiguous and transverse to the transverse portion 2.1 itself, for the attachment and connection of the membrane 2 to the shell 1 in correspondence with the relative shaping 1.6, so that the membrane 2 can be configured in the different operating positions. Furthermore, contiguous to each transverse portion 2.1, on the opposite side with respect to the apex 2.5, the membrane 2 comprises an angled portion 2.2 suitable, due to its structure, to change angle and concavity during the different phases of use and, therefore, consistent with the use of the container 100, as visible in FIG. 1E.

Advantageously, as seen in FIG. 2D, in further embodiments but not limitation, the membrane 2 comprises a second apex 2.5.2, contiguous and transverse to the portion 2.1 itself, in particular, positioned adjacent and facing the same side as the apex 2.5.

Furthermore, again advantageously, the membrane 2 further comprises a third apex 2.5.1, transverse to the portion 2.1, positioned between the first and second apexes 2.5 and 2.5.2, but facing in the opposite direction of the aforementioned first and second apexes 2.5 and 2.5.2.

In particular, the above-mentioned design of membrane 2 is intended to improve the tightness of membrane 2 itself.

In detail, it is through the ring 4 and the shaping 1.6 that the membrane 2 is calibrated. In particular, since the ring 4 and the shaping 1.6 are substantially rigid with respect to the membrane 2, they press it at the transverse portion 2.1, calibrating it and advantageously creating a seal between the parts.

Advantageously, as mentioned above, the membrane 2 is bi-stable, in fact it has at least two operating positions with respect to the shell 1: an initial position 2p1 (FIG. 1A) and an end-use configuration 2p2 (FIG. 1C); in addition to these two positions, advantageously, the membrane 2 could be in any other intermediate position between them (FIG. 1B).

In the first configuration 2p1, the membrane 2 is made with a shape such that it coincides with the shape of the shell 1 when turned upside down. Said membrane 2 is placed with opposite concavity with respect to the shell 1, structurally realising a hollow chamber A suitable for the containment of liquids, gases or any other substance to be dispensed. It should be noted that several substances or mixtures of substances, such as, by way of example, air and/or other gases, to be dispensed at the same time, can also be placed in said hollow chamber A.

In particular, when the container 100 is in the second end position configuration 2p2 (FIG. 1C), since the membrane 2 is shaped in such a way as to adhere to the shell 1, when the membrane 2 is compressed by the plunger 3, the same collapses until it substantially matches the shell 1, in the direction of the compression so as to eject all the contents present in the chamber A. Again advantageously, the membrane 2 is made to coincide perfectly, when collapsed, with the geometry of the inner surface of the shell 1, so as to eject all the liquid present in the chamber A.

Advantageously, as mentioned above, the membrane 2 may also be of the bi-stable type and thus not need to be held in place by the plunger 3 and to be squeezed so as to fit the shell 1

Further, in order to eject the substance contained in chamber A, the plunger 3 must exert a force on the membrane 2 that varies depending on the thickness, elasticity, hardness, tensile strength and shape of the membrane 2 and the back pressure realised by the dispensing portion 1.1.

More specifically, advantageously, using a membrane 2 with a thickness profile adjusted so that the force required to move the plunger 3 is within the prescribed range also allows the thickness profile of the membrane 2 to be designed so that the minimum and maximum force to move the plunger 3 is within the prescribed range.

Advantageously, in the end position 2p2 (FIG. 1E), when the membrane has been deformed by the plunger 3, totally on the shell 1 of the container 100, the angled portion 2.2 of the membrane 2 will assume a certain radius which, if it is coincident with that of the section 1.4 of the shell 1 will not structurally allow the creation of a space G between the two and therefore nothing can be trapped between them (FIGS. 1D and 1E).

In addition, the membrane 2 is designed in such a way that it is deformed, only as an example, by the plunger 3, to move at least between the two configurations 2pi and 2p2.

In FIG. 1B, membrane 2 is shown as a partially deformed membrane. Particularly, depending on the diameter of the membrane 2, the depth of the movement of the plunger 3 to squeeze the contents out of the chamber A and the back pressure generated in the chamber A during the forward movement of the plunger 3, the membrane 2 tends to fold. For the reasons mentioned above, it is essential to adjust the geometry of the plunger rod 3 to allow the necessary space to accommodate the folding of diaphragm 2. In fact, if such an adjustment of the geometry of the plunger 3 is not made, the force to move the plunger 3 could rapidly increase to the point of making it impossible to use the syringe.

Generally, a key element is that in any disposable syringe it is important to expel all contents at the end of the dispensing process.

In a syringe with a membrane, it is intuitive to match the size and shape of the plunger with the shape of the shell. On the other hand, this attempt to match the shape of the plunger with the shape of the shell to squeeze the liquid may be at odds with the need to provide a space to accommodate the folds of the membrane when the plunger advances to expel the liquid.

More specifically, the stability of the contents of the container, for example the pharmaceutical formulation contained in chamber A, depends on gas exchange and the tendency of the contents to migrate from chamber A to the external environment. This leads to a disadvantage because, polymeric and elastomeric materials are permeable to gases and the substance or formulation. Therefore, if shell 1 and membrane 2 are not thick enough, the product contained in chamber A may not achieve the desired stability.

Furthermore, stability is also a function of the surface area/volume ratio of chamber A. Of all geometric shapes, the spherical shape is the most efficient as the smallest surface area can contain the maximum volume. Any deviation from the spherical shape of chamber A leads to a reduction in the stability of the contents compared to the spherical shape, provided that the other variables and the thickness of the membrane and shell remain unchanged.

For the above reasons, nowadays, in order to reduce production costs, reduce the carbon footprint and reduce leachates (additives and volatiles contained in the polymer that are released in the formulation) there is a tendency to reduce thickness, so the spherical shape may help, but the elastomeric membrane, being more permeable, needs a certain thickness to maintain the stability of the formulation contained in the A chamber. The minimum thickness for elastomeric membranes used on the market today should be 0.2 mm.

In addition, nowadays most syringes, whether pre-filled or single-use empty, are equipped with a standard luer port on which a needle with a hub can be mounted. In more detail, the standard luer port has a small hole to allow the passage of the formulation contained in chamber A when the plunger is pushed to eject the substance contained in the container.

A known issue of the related art is that when the membrane is lying in its initial position or initial configuration 2P1, it is not easy to fill chamber A of the syringe through such a small hole because the air that is displaced has to escape through the same hole.

To facilitate filling, the membrane 2 can be collapsed by applying vacuum to the dispensing organ or port or by applying compressed air to the other side of the membrane 2. In this collapsed form of membrane 2, the volume of chamber A is reduced to zero and thus chamber A can be filled without the need to vent air, because there is no air in chamber A when membrane 2 is collapsed. This has the advantage of greatly reducing filling times, and filling chamber A when it is empty also reduces the chances of the substance escaping.

Advantageously, as can be seen in FIGS. 1A-1E, the container 100 also has a ring 4 located in correspondence with the first portion 1.2 of the shell 1, and such that it extends in a direction opposite to the dispensing organ 1.1.1 of the shell 1.

In particular, the ring 4 has dimensions such as to fit at least partially within the first portion 1.2 of the shell 1.

The portion 1.2 of the shell 1 and the corresponding area of the ring 4 are coupled using various methods having the purpose of joining them; such methods may be, for example, welding, gluing, mechanical coupling, interference, etc.

The ring 4, as visible in FIGS. 1D and 1E advantageously comprises a first projecting segment 4.1 of a shape substantially complementary to the transverse portion 2.1 of the membrane 2 and facing inwards from the chamber A of the shell 1, substantially from the side that will contact the plunger 3.

Furthermore, contiguous to and on the opposite side with respect to the first projecting segment 4.1, the ring 4 further comprises a flat segment 4.3 complementary to and such as to contact the apex 2.5 of the membrane 2.

Additionally, the ring 4, in a position opposite the end facing the second portion 1.1 of the shell 1, comprises a support 4.5 for the finger, which is in contact with the plunger 3 to facilitate the use of the container 100 itself. Advantageously, the ring 4, is capable of guiding the sliding of the plunger 3 inside the container 100, possibly also blocking its rotation.

More in detail (FIG. 1D), the ring 4 is apt to accommodate within it the plunger 3, in particular during its movement in the direction of the shell 1, and to press and block the transverse portion 2.1 of the membrane 2 at the shaping 1.6 of the shell 1, holding it in position. Further, in other preferred embodiments, in order to create sealing between the shell 1 and the membrane 2, the membrane can not only be compressed by the ring 4, as described above, but advantageously, it can be sealed by further welding/joining.

As can be seen in FIG. 1D, the first projecting segment 4.1 and the flat segment 4.3 of the ring 4 are shaped in such a way that they simultaneously and respectively press the apex 2.5 and the transverse portion 2.1 of the membrane 2 inside the shaping 1.6 of the shell 1, holding the membrane 2 in place and acting as a seal thereof. Advantageously, the dimensions of ring 4, membrane 2 and shell 1 are such that the pressure exerted by ring 4 must not exceed the elastic modulus of membrane 2.

Advantageously, the plunger 3, as visible in Figures TA, 1B and 1C has a substantially ogival shape and comprises a head 3.1 apt to push and compress the membrane 2 inside the shell 1 for the ejection of the substance contained in the container 100, during its use, and a body 3.2 of oblong shape apt to be placed in correspondence with the ring 4 and apt to transmit the force on the membrane during the use of the container 100.

Said plunger 3 may be made, for example, of plastic, and/or by moulding and/or other fabrication techniques.

In particular, the shape of the head 3.1 of the plunger 3 is substantially complementary to that of the shell 1, so that, during operation of the container 100, the same can push the membrane 2 causing it to completely overlap, since it has the same geometry as the interior of the shell 1, causing the substance contained therein to completely spill out.

The geometry of the head 3.1 of the plunger can be realised to be able to adjust the force required to deform the membrane.

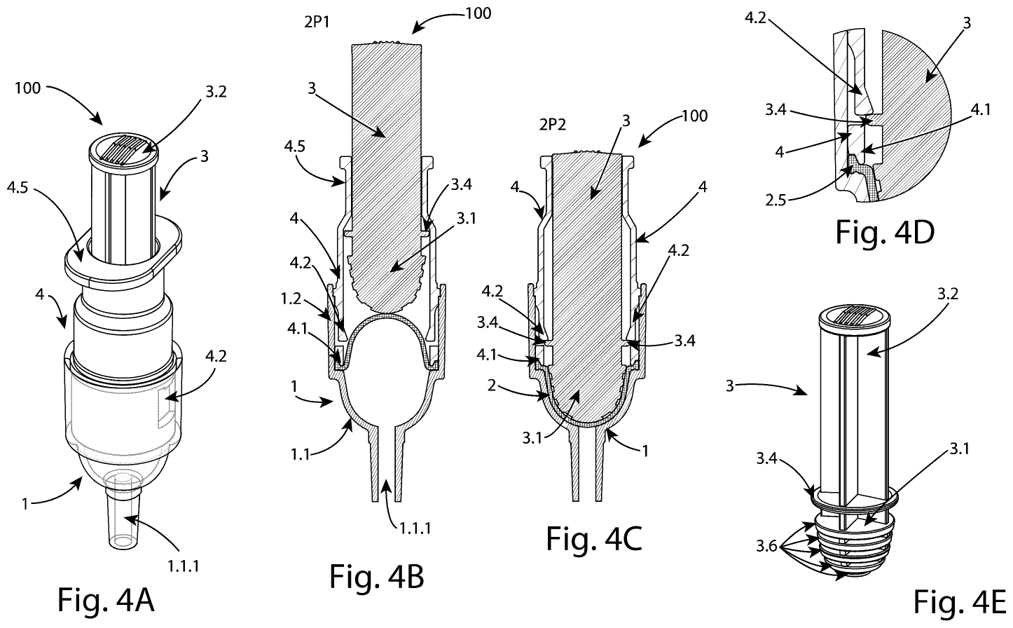

Advantageously, the head 3.1 of the plunger 3, in preferred embodiments as seen in FIG. 4E may have a plurality of concentric slots 3.6 to reduce the weight of the plunger 3 and to, advantageously, lighten the weight of the plunger itself and adjust the force required for deformation of the membrane. In addition, plunger 3 is triggerable via a finger/thumb of the user.

The shell 1, in a further embodiment, as visible in FIG. 2F, substantially in correspondence with the shaping 1.6, as described above, includes a protruding portion 1.3 which, due to its geometry, is apt to improve the seal obtained with the transverse portion 2.1 of the membrane 2. In particular, advantageously, the geometry of the protruding portion 1.3 is projecting and facing in the direction of the transverse portion 2.1 of the membrane 2. Preferably, said projecting portion 1.3 has a curved profile.

Further, the shell 1, as visible in FIG. 3B, may comprise grooves 1.7 facing in the direction of the chamber A, which are intended to allow the substance contained in the container to flow out, to prevent it from being trapped in the points G of disorder, as described above and as visible in FIG. 3A.

In a further embodiment, as visible in FIGS. 4A to 4D, the ring 4 comprises a second protruding lever segment 4.2 which, after use of the container 100, contacts the plunger 3, in such a way as to secure it to the shell 1, making it impossible to remove it. In detail, the plunger 3 has, along the body 3.2, one or more rings 3.4 to prevent reuse of the container 100 after use by preventing the container from returning to the starting operating position 2pi and, therefore, acting as a coupling to the second protruding segment 4.2 lever of the ring 4.

Advantageously, such second protruding lever segment 4.2 structurally results on the inside from the first portion 1.2 of the shell 1, so as to be inaccessible from the outside. Advantageously, this makes tampering with the protruding element 4.2 impossible.

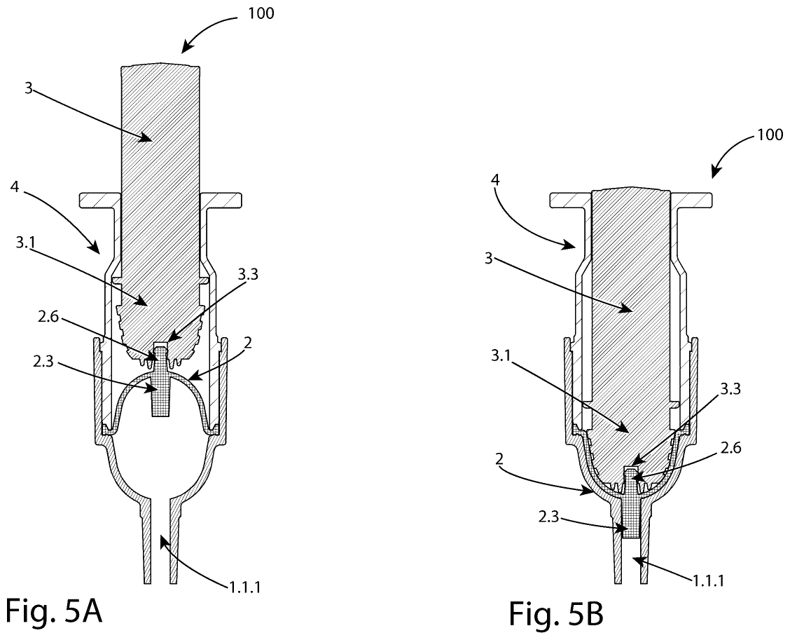

In further preferred embodiments of the container 100, as visible in FIGS. 5A and 5B, the membrane 2 and the plunger 3 comprise, respectively, a coupling 2.6 and a hole 3.3; in detail, the geometry of the coupling 2.6 and the hole 3.3 is such as to allow the coupling 2.6 to be locked inside the hole 3.3, not allowing it to be unlocked. This embodiment may be used to allow the membrane 2 to move according to the first operating position, i.e. in the opposite direction to the concavity of the shell 1, creating a depression in the chamber A. In particular, this form of embodiment allows the substance to be sucked into the chamber A of the shell 1, in the event that the substance is to be sucked from the container 100, for example, by means of a movement in the opposite direction to that of the dispensing given by the plunger 3.

Additionally, the membrane 2 may have a protrusion, in particular a cap 2.3, facing in the opposite direction of the coupling 2.6, such that it can be used to squeeze the liquid trapped in the dispensing organ 1.1.1, ensuring that all of the substance is ejected from the container 100.

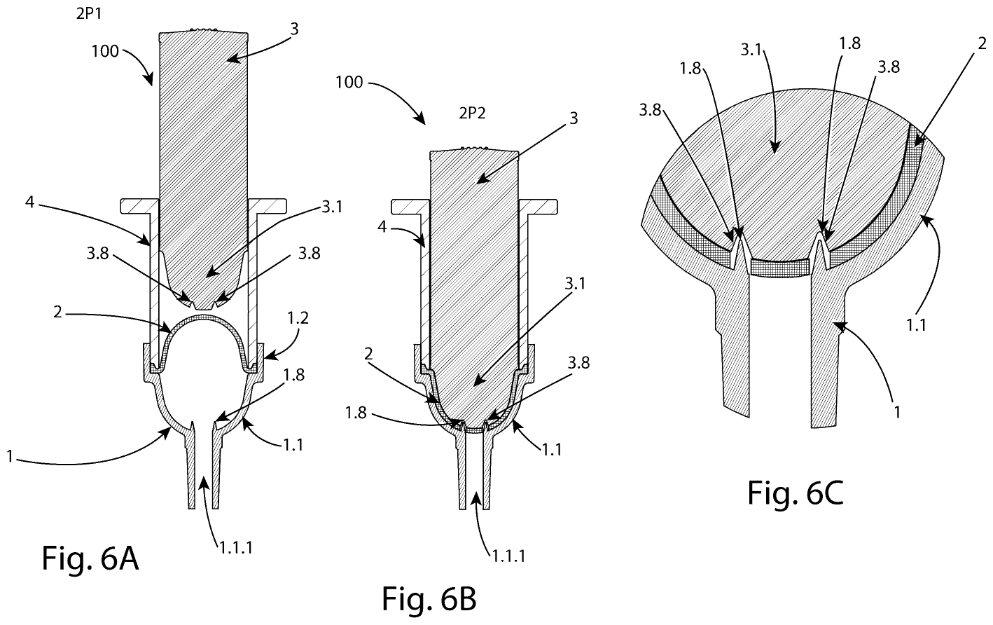

In further detail, in a further embodiment, the plunger 3, as visible in FIGS. 6A to 6C, in correspondence with the respective head 3.1 also comprises cavities 3.8, while the shell 1, in correspondence with the said cavities 3.8 has, in its respective inner surface, sharp-edged notches 1.8, for cutting the membrane 2, following the use of the container 100, in particular, in its end-use The presence of the aforementioned notches 1.8 advantageously allows the container 100 to be prevented from being reused as they are capable of permanently puncturing the membrane 2.

Alternatively, in further embodiments, the series of sharp-edged notches can be positioned on the head 3.1 of the plunger 3; in this case, the shell 1 has holes to allow the membrane 2 to stretch and break on the aforementioned notches of the head 3.1, to prevent reuse of the device.

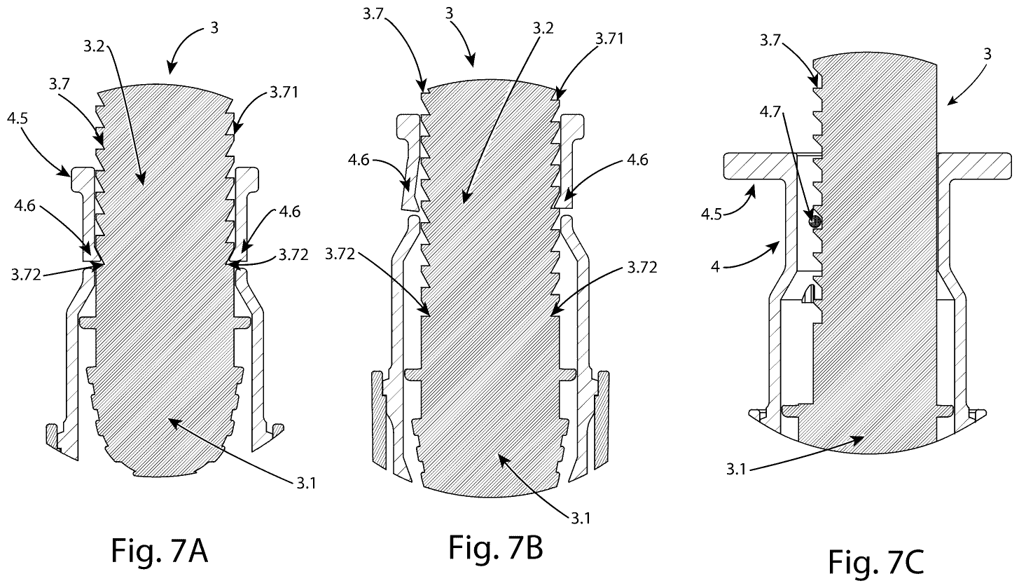

In a further embodiment of the container 100, as visible in FIGS. 7A and 7B, the ring 4 may comprise a plurality of hooks 4.6 projecting and facing into the container 100 and suitable for contacting a toothed profile comprising a plurality of teeth 3.7 and 3.71 which are substantially arranged on the body 3.2 of the plunger 3.

In detail, at the body 3.2, the plunger 3 comprises a toothed profile, wherein the teeth 3.7, 3.71 are configured to act as a control relative to the dose of substance to be ejected from the container 100. In detail, advantageously, the distance between the teeth corresponds to a unit of volume of substance to be ejected and is such as to maintain said volume constant during ejection: the more teeth there are, the more clicks there are and, for this reason, this type of configuration could be used, advantageously, to have tactile and/or auditory feedback on the flow/dose of substance ejected. Additionally, again advantageously, the aforementioned toothed profile, in preferred designs, is suitable for removing air present in chamber A, for example after filling the container. In particular, as the toothed profile exerts pressure on the membrane 2 it is such as to compress it, removing the air present in the chamber A and locking it in position, as explained above, to advantageously allow the container 100 to be used correctly.

If the teeth 3.7, 3.71 of the toothed profile are of the sharp-edged type, as visible in FIGS. 7A-7C, they prevent the movement of the plunger 3 in the opposite direction to the direction of ejection, such movement possibly caused, for example, by pressure in chamber A.

The teeth 3.7, 3.71, in another embodiment, can be of the rounded type, also allowing the movement of the plunger 3 in the opposite direction with respect to the direction of ejection, only creating a feedback system that allows the user a better precision in dosing the substance contained in the container 100.

In order to increase the overall number of clicks the teeth 3.7 and 3.71 can be offset between one side and the other of the body 3.2 of the plunger 3, as visible in FIGS. 7A and 7B. To avoid stress on the hooks 4.6, the body 3.2 has aligned teeth 3.72 when in position 2P1.

Another embodiment of the ring 4, shown in FIG. 7C, the ring 4 has a transverse through-hole which allows the passage of a wire 4.7, realising a mechanism similar to that discussed in the previous embodiment.

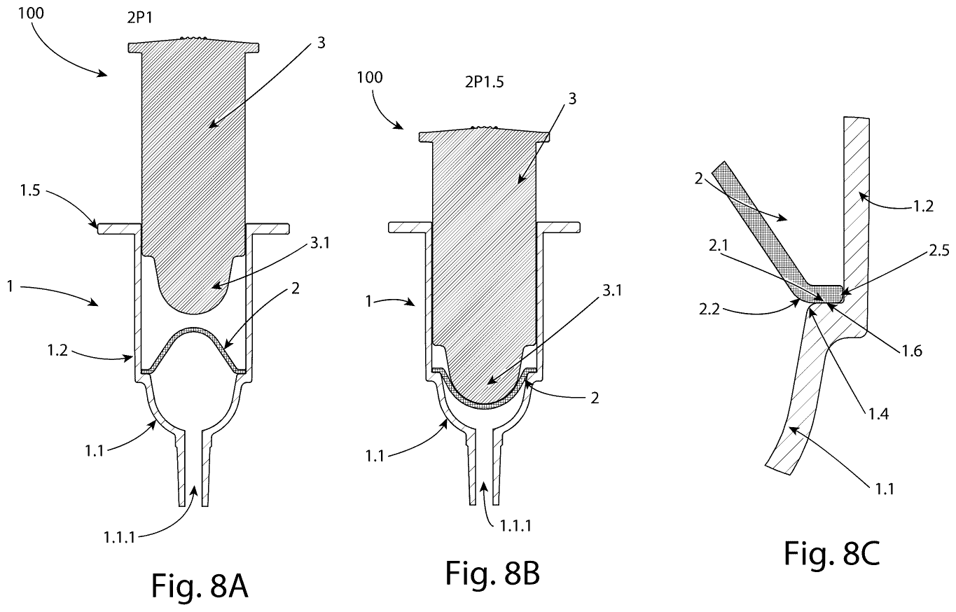

A further embodiment, shown in FIGS. 8A to 8C, the shell 1 has the first portion 1.2 more elongated than in the previous embodiments, so as to present a support section 1.5, suitable to act as a support for the fingers of a user.

By way of example only, said support section 1.5 is projecting in the direction of the outside of the container 100.

Advantageously, in the embodiment under analysis, the ring 4 is not present and the membrane 2 is coupled to the shell 1 between the shaping 1.6 and the transverse portion 2.1, for example, by a heat welding method.

In this embodiment, the membrane 2 after arriving at the intermediate position 2P1.5 is stretched by the movement of the plunger 3 itself; this allows the membrane 2 to adhere better to the inner surface of the shell 1 causing less liquid to remain inside after use.

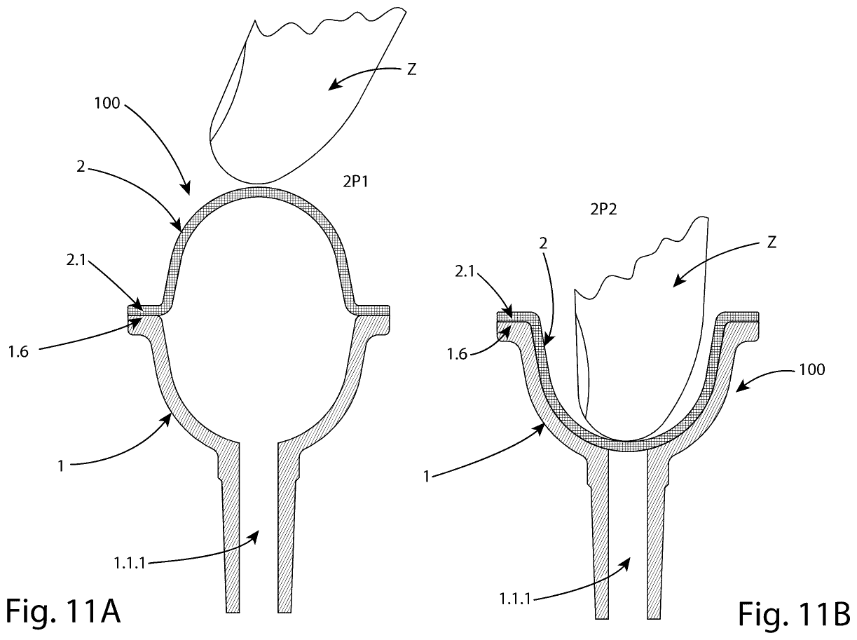

A further embodiment of the container 100, as visible in FIGS. 11A and 11B, is characterized by the absence of the plunger 3. The membrane 2 and the shell 1 are coupled using the methods described above such as, for example, heat welding or gluing. Further, again in the embodiment form under analysis of the container 100, it may have the ring 4 so as to form the seal between the membrane 2 and the shell 1. The advantage of this embodiment is that the membrane is made with a bi-stable shape, which allows the membrane to remain in place even when the user removes the finger Z after use.

Further, in an embodiment of the container 100, not shown in the figures, the membrane 2 is deformable by means of a spring.

In particular, this embodiment is advantageous for the following reasons.

Shell 1, plunger rod 3, ring 4 and dispensing organ 1.1.1 all occupy a certain volume. Plunger rod 3 and ring 4 should be at least twice as long as the stroke required to move the entire volume contained in chamber A. Thus, by providing for a plunger rod 3 to eject the contents from chamber A, it is necessary for the container 100 to provide at least 3 times the volume of chamber A.

For these reasons, advantageously, the work of ejecting the contents can also be carried out with the aid of a compressed conical spring which, once released, can expand to eject the contents from chamber A, without contributing significantly to the volume occupied by the container 100. Such a spring-activated container 100 will occupy a volume slightly greater than that of the spherical chamber A, resulting in a significant reduction in volume compared to that required by the container 100 with the plunger rod 3.

Advantageously, the spring could be shaped and designed in such a way that, when extended, it assumes the contour of the membrane 2 in its stable position, so that the contour of the extended spring helps to eject the entire contents of chamber A. This leads to a reduction in secondary volume and a reduction in secondary and tertiary packaging material and transport costs. This volume saving is highly appreciated in the case of vaccines, which are transported in the cold chain and the space occupied by the syringe is an issue.

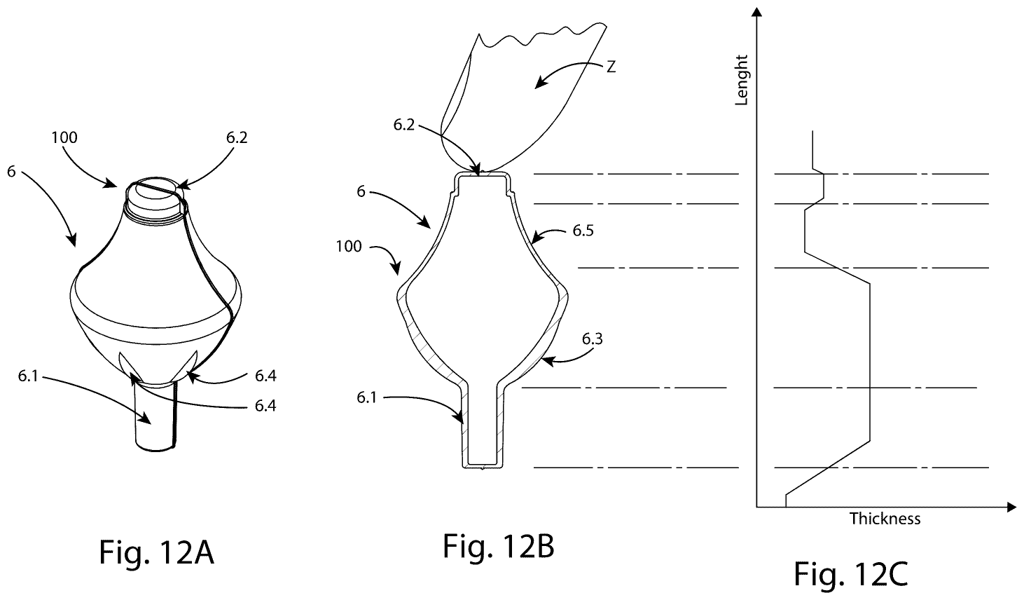

A further embodiment of the container 100 is shown schematically in FIGS. 12A and 12B. In detail, the container 100, unlike the previous embodiments, does not include any membrane and is made in one piece 6 using, for example, the BFS (Blow-Fill-Seal) system.

In particular, the container 100 has a first zone 6.1 suitable for allowing the substance to exit, which may have a threaded connection or a luer insert or various shapes suitable for dispensing/ejecting the substance contained within the container 100.

Additionally, in a position opposite the first zone 6.1 there is a second zone 6.2 from which, advantageously, the filling of the container 100 can take place during the respective creation process and from which the compression of the container through, for example, the finger Z takes place.

As is particularly visible in FIGS. 12B and 12C, the advantage of this embodiment is inherent in the fact that, structurally, the container 100 comprises two different thicknesses. In particular, a first area 6.3 having a first thickness, which is placed substantially above the first zone 6.1 and, a second area 6.5 having a second thickness, which is less than the first thickness, which is placed directly below the second zone 6.2 of the container 100.

In particular, this difference in thickness between the parts of the container 100, is also accentuated by possible reinforcements 6.4 placed radially on the first area 6.3 and around the first zone 6.1 of the container. In particular, this difference in thickness means that, the zone of lower thickness, which is placed in correspondence with the second area 6.5, can deform in the direction of the interior of the container 100, functioning in a similar way to the mechanism of operation of the membrane 2, as described in the previous embodiments, in which the membrane 2 is apt to collapse inside the shell 1. Variation in the thickness of the container is enabled by controlling the thickness of the parison, which follows a profile as described in FIG. 12C.

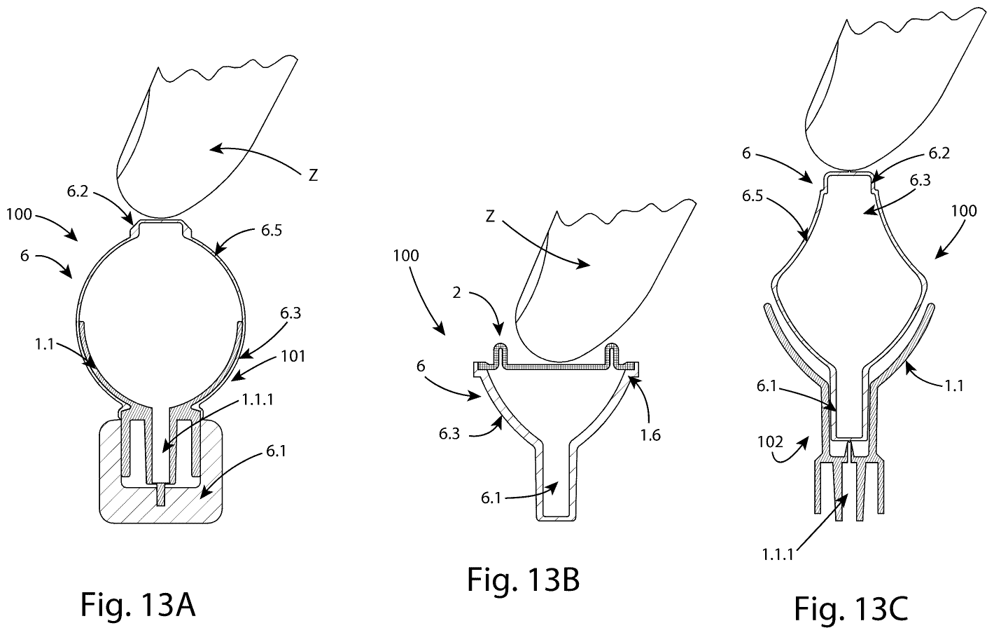

Two further embodiments are illustrated in FIGS. 13A and 13B, in which the container is fabricated using the BFS process by adding an insert during the manufacturing process.

In FIG. 13A, the shell-shaped insert 101 is inserted during the BFS process and, in particular, before the mould is closed. The shell 101, similarly to what has been described in previous embodiments is composed of a containment portion 1.1, for example hemispherical, and a dispensing organ 1.1.1 to which various dispensing systems can be applied.

The first zone 6.1 of the container 100 made of BFS serves as the cap of the container 100, which must be removed before use to allow ejection of the substance contained therein. Following the insertion phase of the shell 101 as described, the filling phase of the container 100 takes place through the opening located in the second zone 6.2, which is then sealed by the subsequent closing of the mould heads to form a closed container.

The advantage of this form of embodiment over the previous form of embodiment, described in FIGS. 12A and 12B, is that a drastic change in thickness between the areas 6.3 and 6.5 is not necessary since the shell 1 acts as a reinforcement of the area 6.3. Advantageously, area 6.5 is apt to collapse when crushed, similar to that described above.

FIG. 13B shows the other embodiment of the container 100, in which the insert that is inserted during the BFS process is the membrane 2. The container 100 is made in open BFS, and when the latter is still malleable the membrane 2 is incorporated and/or welded onto the moulding 1.6.

Finally, a further embodiment, depicted in FIG. 13C, shows the container 100 comprising an outer rigid shell 102 and a closed container 6 made from BFS. In correspondence with the dispensing organ 1.1.1 of the shell 102 there is a coupling system designed to connect the dispensing organ 1.1.1 with the first zone 6.1 of the container 6. In particular, the container 100 in order to be used according to the aforementioned embodiment, must be activated, i.e. the container 6 is pushed towards the shell 102 and the portion 1.1 comes into contact with the area 6.5 of the container acting as a reinforcement.

The container 100 functions substantially as follows. As mentioned above, in the first configuration 2P1 in which the container 100 is closed (container 100 full, prior to ejection of the substance) the membrane 2, having substantially the same geometry as the shell 1, is placed with opposite concavity with respect to the same, structurally generating the hollow chamber A containing the substance to be dispensed. Subsequently, the plunger 3, by means of an external pressure acting in the direction of the shell 1 itself, begins to gradually compress the membrane 2. At this point, through the compression of the plunger 3, the membrane 2 gradually changes concavity while simultaneously pushing the substance contained within the chamber A towards the dispensing organ 1.1.1. Chamber A begins to empty until the membrane 2 collapses, adhering substantially to the inner surface of the shell 1.

In particular, when the container 100 is in the second end-use configuration 2P2, as the membrane 2 is shaped to coincide with the shell 1, the membrane 2 is compressed by the plunger 3, and collapses back to substantially match the shell 1, in the direction of compression so as to eject all the contents present in the chamber A.

Advantageously, in the end position 2P2, when the membrane has been compressed by the plunger 3 totally on the shell 1 of the container 100, the angled portion 2.2 of the membrane 2 will assume a certain radius which, if it is coincident with that of the section 1.4 of the shell 1, will not structurally permit the creation of a space G between the two and therefore nothing can be trapped in this position.

Today, there are two main methods for producing elastomeric membranes: injection moulding and vulcanisation. Although the hemispherical membrane remains stable in two positions, the position in which it has the least internal tension is the position in which it is unmoulded by the injection mould or vulcanisation mould.

This state of minimum internal stress can be further improved by annealing the membrane after moulding.

In this state of minimum stress, the elastic membrane will not form creases but will faithfully maintain the contours that the mould imprints on the membrane during the moulding process. We call this the first stable position of the bistable membrane.

When this membrane is turned into its second stable position, stresses are generated in the membrane, because the inner part of the membrane tends to compress and the outer part of the membrane tends to stretch. As a result of these stresses, dents, dimples and ridges appear on the surface of the membrane.

Thus, according to the present application, in the initial position or first use configuration 2P1 of the container 100, when the chamber A is fully stretched, if the membrane 2 is already turned to its second stable position and the plunger 3 is pushed to eject the contents of the chamber A, the membrane 2 returns to its first stable position in which it was unstretched. Since this is the position where residual stresses in membrane 2 are minimal, it tends to assume its original profile. Thus, without dents, dimples and ridges, the membrane will expel all the liquid contained in chamber A. In fact, in the absence of surface irregularities, membrane 2 tends to adhere to the surface of shell 1 even when the shape of plunger 3 does not correspond exactly to that of shell 1.

Advantageously, the following table gives limit values, as an example, to ensure that the maximum amount of substance content in chamber A can be squeezed through the dispensing apparatus 1.1.1, for a container filling volume of 0.2 ml to 100 ml.

The thickness of the membrane 2 and the surface area of the membrane 2 should be such that the force required for squeezing remains within the desired limits.

The following table gives the maximum and minimum values for membrane thickness and surface area:

| | LIMIT VALUES | |
|---|---|---|
| | (Maximum and | |
| PARAMETER | minimum) | COMMENT |
| One or more of the | From 5 mm to 70 | If we assume that the membrane is |
| dimensions of a square or | mm | square or a rectangle, the dimension of |
| rectangular membrane | | the membrane of the smallest container |
| (not the thickness) | | with a filling volume of 0.05 ml could |
| | | be 5 mm × 5 mm |
| If the shape of the | From 5 mm to 70 | |
| membrane is round, its | mm | |
| diameter must be | | |
| between: | | |
| Surface area of the | From 10 mm2 ti | If we assume that the membrane is |
| membrane in contact with | 6000 mm2 | circular, the diameter could be between |
| content of chamber A | | 5 mm and 70 mm. The smallest |
| | | container with a membrane diameter of |
| | | 5 mm could have a filling volume of |
| | | 0.1 ml. |
| Membrane perimeter if | From 10 mm to 150 | Considering that the 100 ml syringe |
| the membrane is not | mm | might have a 70 mm diameter |
| round, rectangular or | | membrane. |
| square | | |
| Membrane thickness | From 0.1 mm to 5 | The thickness may be different if the |
| | mm | syringe is designed to aspirate the |
| | | liquid |
| Membrane hardness in | From 10 to 150 | For container filling volume between |
| Shore A or D | | 0.05 ml and 100 ml |
| Diametro dello stantuffo | From 3 mm to 70 | For syringe filling volume between |
| se ha circonferenza | mm | 0.05 ml and 100 ml |
| rotonda | | |
| Plunger size if not round. | 3 mm to 100 mm | |
| Tensile strength of | 1 Mpa to 85 Mpa | For syringe filling volume between |
| membrane material | | 0.05 ml and 100 ml |
| Membrane material | Any Polymer, | |
| | Elastomer, TPE, | |
| | TPV complying | |
| | with current | |
| | regulations and | |
| | compatible with the | |
| | intended content. | |
| Materials that can be used | Polypropylene, | Shore hardness depends on filling |
| with the BFS process | polyethylene, EVA | volume A higher shore hardness is |
| | copolymer. | required if the syringe is designed to |
| | | aspirate liquid. |
| If the circumference of | 0.2 - 5 | |
| the membrane is round, | | |
| the ratio of its height to | | |
| the radius (h/r) is | | |

Generally, the production method for the realization of the container 100 comprises one or more of the following steps:
Making the parts of the container, in detail: shell 1, membrane 2, plunger 3 and ring 4, by one or more of the following processes: blow moulding, injection moulding, compression moulding, BFS, thermoforming or 3d printing.
Assembly of the above parts in a suitable/sterile environment;
Secondary packaging of the container in a suitable environment
Terminal sterilization of the container using one or more of the known sterilization methods.

The container 100 is realizable by means of different methods; the fundamental element of the production process is that it is realised in such a way that a good seal is advantageously created between the membrane 2 and the shell 1. Advantageously, this optimal seal between the two elements is created by the pressure exerted by the ring 4.

Alternatively, to maintain the seal between the shaping 1.6 of the shell 1 and the membrane 2, a layer of glue can be advantageously provided.

Yet another method to achieve a tight seal between the two elements is to heat the shaping 1.6 and the transverse portion 2.1 and then apply external pressure to it (FIG. 8C).

Another method would be to make the 2.5 portion of the membrane 2 so that it interferes with the 1.2 portion of the shell 1.

With regard to injection and/or compression moulding, an innovative step would be to advantageously control the tooling cooling process in such a way that the weld zone, such as for example the shaping 1.6, and the cross-sectional portion, such as for example the transverse portion 2.1, can remain relatively warm compared to the rest of the membrane 2 and the rest of the shell 1.

In detail, after such controlled cooling, when the shell 1 and the membrane 2 have been formed, the transverse portion 2.1 of the membrane 2 and the shaping 1.6 of the shell 1 still have a relatively higher temperature than the other parts of the container 100, by way of example only a temperature above 60° C., so advantageously, the membrane 2 when coupled to the shell 1 welds without the use of additional mechanical instrumentation or without the need to heat the parts prior to coupling.

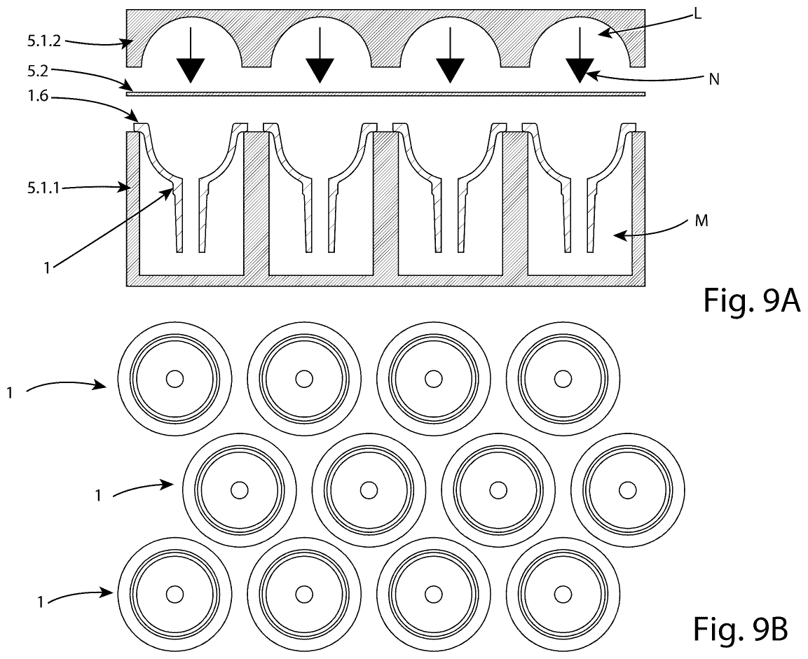

A further innovative production method could be to arrange several shells 1 in a matrix, as shown in FIG. 9A, using a suitable nest 5.1.1. In detail, a heat-sealable film 5.2 is then placed against the top of the shells 1 being manufactured, wherein only the shaping 1.6 of the shell 1 and the film 5.2 are heated.

Advantageously, one or a combination of several known methods may be used to heat said heat-sealable film 5.2, such as, but not limited to, induction, hot air blowing, the use of infrared (IR) lamps, by convection or by any other means/methodology suitable for heating.

After heating, the mould can be pressed against the still malleable moulding die of the shells 1 to weld the film 5.2 with the shaping 1.6 of the shell 1.

A pressure difference is applicable both at the level of the nest 5.1.1 and at the cavity L of the mould 5.1.2 which is placed above the nest 5.1.1. In this way, the film 5.2 will deform to form the relatively flexible membrane 2.

With particular reference to FIGS. 12A, 12B and 12C, another method that can be used to make the container 100 is the “Blow Fill Seal” (BFS), a manufacturing process that consists of extruding a tubular parison of suitable polymer to mould the rigid shell 1 and membrane 2, filling the container 100 with the substance and sealing the container from the top of the mould in the zone 6.2. Advantageously, as mentioned above, the innovative step of the “Blow Fill Seal” process consists in moulding the part of the shell with a greater thickness to make the shell 1 relatively rigid and the part of the membrane 2 with a lower thickness to make it more flexible using, for example, automatic parison thickness control.

The container and techniques thus conceived and illustrated herein are susceptible to numerous modifications and variations, all within the scope of the present disclosure.

Furthermore, all details may be replaced by other technically equivalent elements.

Finally, the components used, as long as they are compatible with the specific use, as well as the dimensions, may be varied according to requirements and the state of the art.

Where features and techniques mentioned in any claim are followed by reference marks, those reference marks have been included for the sole purpose of increasing the intelligibility of the claims and, consequently, those reference marks have no limiting effect on the interpretation of each element identified by way of example by those reference marks.

1. A container for ejecting a substance, comprising:
a hollow shell for containment and ejection of said substance, including:
a containment portion comprising a dispensing organ of oblong shape, and
a membrane, connected to said shell, and
said container further comprising, in a position contiguous to and transverse with respect to said containment portion, a shaping element for connecting said membrane, so that said membrane can move from a first operating position, in which the membrane has a central portion in a position opposite to the containment portion of the shell, creating with the containment portion a chamber for containment of said substance, to a second operating position, wherein the central portion of the membrane collapses within said containment portion of said shell, so as to eject said substance present in the chamber through said dispensing organ,
wherein said membrane is of a bi-stable type satisfying at least one of:
a thickness between 0.1 mm and 5 mm;
a membrane hardness in Shore A or D between 10 to 150;
a tensile strength of the membrane from 1 MPa to 85 MPa; or
a material comprising any polymer, elastomer, thermoplastic elastomer (TPE), thermoplastic vulcanizates (TPV), polypropylene, polyethylene, or ethylene-vinyl acetate (EVA) copolymer;
wherein said membrane independently maintains its own operating position between said first operating position and said second operating position; and
wherein, depending on the thickness of said membrane, an intensity of a compressive force applied on said membrane and a bistability of said membrane vary.

2. The container as in claim 1, wherein said membrane is calibrated with a geometry of said containment portion of said shell, so as to adhere to and be compressible in a direction of said shell, for ejecting said substance contained in said chamber.

3. (canceled)

4. (canceled)

5. The container of claim 1, wherein said membrane is a prolate or oblate spheroid or other form suitable for containing said substance and collapsing into the shell.

6. The container as in claim 5, wherein said shell comprises a first set of sharp-edged notches for perforating said membrane in the second operating position, for ejecting said substance.

7. (canceled)

8. The container of claim 1, wherein said dispensing organ of said containment portion of said shell is connected to a dispensing accessory.

9. The container of claim 1, wherein said shell comprises a support section for fingers of a user.

10. The container according to claim 6, further comprising a movable activator device having a shape corresponding to the shape of said shell, the movable activator device having a head adapted to push and compress said membrane inside said shell in order to pass from said first operating position to said second operating position, and a body adapted to transmit compression on said membrane during use of the container.

11. The container as in claim 10, wherein said head of said movable activator device comprises a plurality of concentric slots for reducing a weight of said movable activator device and for regulating a force necessary for deformation of said membrane.

12. The container according to claim 10, wherein said head comprises cavities for perforating said membrane by means of said first set of sharp-edged notches present on an inner surface of said shell, preventing reuse of said container.

13. The container of claim 10, wherein said head of the movable activator device comprises a second set of sharp-edged notches capable of perforating said membrane after relative compression, preventing reuse of said container.

14. (canceled)

15. (canceled)

16. (canceled)

17. The container of claim 10, further comprising a ring for locking said membrane to said shell, wherein said ring is coupled to said shell and is configured to accommodate the movable activator device for guiding movement of the ring, and wherein said shell comprises a first portion which is tubular and hollow contiguous to said containment portion, and wherein said ring is coupled to fit within the first portion of said shell.

18. (canceled)

19. The container of claim 17, wherein said membrane comprises an edge including a transverse portion terminating in a first apex contiguous with and transverse to said transverse portion.

20. The container of claim 19, wherein said membrane further comprises:
a second apex contiguous and transverse to said transverse portion, wherein said second apex is adjacent to and facing in a same direction as said first apex, and
a third apex transverse to the transverse portion and positioned between the first apex and the second apex, the third apex facing in an opposite direction from said first apex and said second apex to improve a

21. The container of claim 20, wherein said movable activator device further comprises:
along the body of the movable activator device, one or more rings for preventing reuse of the container by preventing the container from returning to the first operating position; and
a projecting element for switching from a rest position corresponding to said first operating position to an activation position corresponding to said second operating position, wherein the projecting element is engaged with at least one of the one or more rings to secure the movable activator device to the shell and prevent reuse of the container.

22. (canceled)

23. The container of claim 21, wherein said one or more rings comprises a projecting segment of a shape complementary to said transverse portion of the membrane and facing inwardly toward said chamber, configured to lock said transverse portion of the membrane at the shaping element of the shell by holding the transverse portion in place to form a seal.

24. The container of claim 23, wherein said ring comprises, in a position contiguous with and opposite to said projecting segment, a complementary flat segment configured to contact said first apex of said membrane.

25. The container of claim 24, wherein the projecting segment and said complementary flat segment of the ring are configured to respectively press the first apex and the transverse portion of the membrane within the shaping element of the shell.

26. The container of claim 23, wherein said ring comprises, in a position opposite to the projecting segment, a support preferably for fingers of a user.

27. A production method for producing the container according to claim 23, the production method comprising:
making parts of said container by blow molding, injection molding, compression molding, Blow-Fill-Seal, thermoforming, or 3D printing, wherein said parts comprise the shell, the membrane, the ring, and the movable activator device;
assembling said parts in a sterile environment;
applying secondary packaging to the container; and
performing terminal sterilization on the container.

28. The production method according to claim 27, wherein making said parts of said container, is performed by injection or compression molding techniques, and wherein, during the production method there is a step of controlling a cooling process of said parts so that the shaping element and the transverse portion each have a higher temperature than the membrane and the shell such that the membrane sticks to the shell.

29. The production method according to claim 27, wherein making said parts of said container is carried out by a “Blow Fill Seal” (BFS) technique, and wherein, during the production method, a rigid portion of the shell is molded with a greater thickness than a remaining portion of the shell, and a flexible portion of the membrane is formed with a lower thickness than the remaining portion of the membrane.

30. The production method according to claim 27, wherein making said parts of said container is carried out by a “Blow Fill Seal” (BFS) technique, and wherein, during the production method, a shell-shaped insert is added to said container before closing a mold, wherein said shell-shaped insert comprises the containment portion and the dispensing organ.

31. The production method according to claim 27, wherein making said parts of said container is carried out by a “Blow Fill Seal” (BFS) technique, and wherein, during the production method, an insert formed of a membrane suitable for being incorporated or welded onto the shaping element of the shell is added while said container is still malleable.

32. The production method according to claim 27 wherein, during the making of said parts of said container, the production method further comprises:
arranging a plurality of shells within a molding die using a nest in which a heat-sealable film is provided against a top of each of the plurality of shells;
heating an edge of each of the plurality of shells and the heat-sealable film by induction, hot air blowing, infrared lamps, or convection;
pressing a mold against the molding die of the plurality of shells while the plurality of shells are malleable such that the heat-sealable film is welded to the shaping element of each of the plurality of shells; and
applying a pressure difference to the plurality of shells or to the dispensing organ on the molding die, such that the heat-sealable film deforms to form a relatively flexible membrane.

33. The production method according to claim 27 wherein, during assembling the parts of said container, the ring is positioned to exert pressure between the transverse portion of the membrane and the shaping element of the shell.

34. The production method according to claim 27 wherein, during assembling said parts of the container, a layer of glue is arranged between the shaping element of the shell and the transverse portion of the membrane, to maintain the seal.

35. The production method according to claim 27 wherein, during assembling said parts of the container, in order to achieve a tight seal between the shaping element and the transverse portion, said shaping element and said transverse portion are heated and pressed together.

36. The production method according to claim 27 wherein, during assembling said parts of said container, ultrasound or infrared techniques are used to weld the transverse portion of the membrane to the shaping element of the shell.

37. A production method, wherein container comprising a movable activator device corresponding to a shape of a shell of the container, a head adapted to push and compress a membrane inside said shell in order to pass from a first operating position to a second operating position, and a body adapted to transmit compression of the membrane during use of the container is filled with a substance to be ejected, wherein a toothed profile of said movable activator device along the body of the container has a pitch between teeth of the toothed profile corresponding to a unit volume of said substance, and wherein said toothed profile of said movable activator device is adapted to compress said membrane, eliminating air present in a chamber of said container so that said movable activator device locks in said first operating position.

38. The container of claim 17, wherein said movable activator device comprises a toothed profile along the body, wherein a pitch between teeth of said toothed profile corresponds to a unit volume of said substance to be ejected, and wherein the ring further comprises a plurality of hooks projecting into the container to contact the toothed profile.

39. The container of claim 17, wherein the membrane and the movable activator device comprise, respectively, a coupling and a hole, such that said coupling is to lock within said hole to enable the membrane to be brought to the first operating position and to aspirate said substance into the chamber.

40. The container of claim 39, wherein the membrane comprises a protrusion facing in an opposite direction of the coupling, wherein said protrusion is configured to expel the substance remaining in the dispensing organ.
